SEQUENCE LISTING (8 sequences)

SEQ ID NO: 1 - protein, 16 aa, Homo sapiens
KGYNYSYKSEMKVRPA

SEQ ID NO: 2 - protein, 19 aa, Homo sapiens
GGWTVFQRRVDGSVDFYRK

SEQ ID NO: 3 - protein, 27 aa, Homo sapiens
KGYNYSYKVSEMKFQRRVDGSVDFYRK

SEQ ID NO: 4 - protein, 18 aa, Homo sapiens
KGYKYSYKVSEMKVRPAK

SEQ ID NO: 5 - protein, 19 aa, Homo sapiens
GGWTVFQRRMDGSVDFYRK

SEQ ID NO: 6 - protein, 27 aa, Homo sapiens
KGYKYSYKGGWTVFQRRMDGSVDFYRK

SEQ ID NO: 7 - protein, 27 aa, Homo sapiens
KGYKYSYKVSEMKFQRRMDGSVDFYRK

SEQ ID NO: 8 - protein, 26 aa, Homo sapiens
KGYKYSYKGGWTVFQRRMDGSVDFYR

(19) United States
(12) Patent Application Publication
(10) Pub. No.: US 2012/0058956 A1
(43) Pub. Date: Mar. 8, 2012

(54) PEPTIDES DERIVED FROM FICOLIN AS SURFACTANTS

(75) Inventors: Mazal Dahan, Mazkeret Batia (IL); Ascher Shmulewitz, Tel Aviv (IL)

(73) Assignee: METAMOREFIX LTD., Rehovot (IL)

(21) Appl. No.: 13/254,209

(22) PCT Filed: Mar. 3, 2010
(86) PCT No.: PCT/IL2010/000179

§ 371 Date: Nov. 8, 2011

(30) Foreign Application Priority Data
Mar. 3, 2009 (US) 61202483

(51) Int. Cl. A61K 38/10; A61K 38/17

(52) U.S. Cl. 514/21.3; 514/21.4

(57) ABSTRACT

Provided are peptides having a sequence that is a subsequence of a ficolin protein and methods of use thereof. The peptide may be a subsequence of human ficolin in which case it is essentially non-toxic. Further provided is a pharmaceutical composition including such peptides. The pharmaceutical composition can include an active ingredient for delivery through a body surface such as skin.

### FIELD OF THE INVENTION

This invention relates to pharmaceutical compositions containing peptidic sequences as trans dermal permeation promoters.

### LIST OF PRIOR ART REFERENCES

The following prior art publications are considered relevant for an understanding of the invention. The publications are referred to herein by their number in the following list.
1. Kaushik S. Krishnan A., Prausnitz M. R., Ludovice P. J., Pharm. Res., 18, 894-896, 2001.
2. Kim Y. C., Ludovice P. J., Prausnitz M. R., J. Cont. Rel., 122, 375-383, 2007.
3. Chen Y. et al. Nat. Biotechnol., 23,405-410,2006.
4. Rothbard J. B., Garlington S., Lin Q. et al., Nat. Med., 6, 1253-1257, 2000.
5. Jin L. H., Balm J. H., Eum W. S. et al., Free Radic. Biol. Med, 31, 1509-1519, 2001.
6. Park J., Ryu J., Jin L. H.,et al., Mol. Cells, 13, 202-208, 2002.
7. Robbins P. B., Oliver S. F., Sheu S. M., Goodnough J. B., Wender P., Lhavari P. A., BioTechniques, 33, 190-194, 2002.
8. Lim J. M., Chang M. Y., Park S. G. et al., J. Cosmet. Sci., 54, 483-491, 2003.
9. Hou Y. W., Chan M. H., Hsu H. R., Liu B. R., Chen C. P., Chen H. H., Lee H. J., Exp. Dermatol., 16, 999-1006, 2007.
10. Glogau R. G., Waugh J. M., poster presentation, 66^{th} annual meeting of the American Academy of Dermatology, February 2008.
11. Gorodetsky R., Vexler A., Shamir M., An J., Levdansky L., Shimeliovich I., Marx G., Exp. Cell Res., 287, 116-129, 2003.
12. Frankenburg S., Grinberg I., Bazal Z., Fingerut L., Pitscovski J., Gorodetsky R., Peretz T., Spira R. M., Skornik Y., Goldstein R. S., Vaccine, 25, 4564-4570, 2007.
13. Wang Y. H., Chen C. P., Chan M. H., Chang M., Hou Y. W., Chen H. H., Hsu H. R., Liu K., Lee H. J., biochem. And Bioph. Res. Conmmun., 346, 758-767, 2006.
14. European Pharmacopea 6.2: Sodium Hylarunate, 07/2008:1472. comprising ficolin subsequences for sequestering cells in connective tissue.
15. International Patent Application PCT/IL2009/001025.
16. International Patent Application 2008/001121.

### BACKGROUND OF THE INVENTION

The human skin is the largest organ of the body, accounting for about 16% of the body's weight. It performs many vital roles as both a barrier and a regulating influence between the outside world and the controlled environment within the body.

There are two main layers of skin. The dermis consists mostly of overlying dermis and the innermost subcutaneous fat layer (hypodermis). The hypodermis is of few mm thick connecting between the dermis and the inner body constituents. The overlying dermis consists of connective tissue and is much thicker than the epidermis (3-5mm). Being composed of collagen fibrils in a mucopolysacharide gel—it is related to as perameable for most hydrophilic molecules. This layer supports the skin and is responsible for the elasticity of it. It is responsible for the skin's pliability and mechanical resistance and is also involved in the regulation of body temperature. The dermis supplies the avascular epidermis with nutrients and contains sense organs for touch, pressure, pain and temperature (Meissner's corpuscles, Pacinian corpuscles, free nerve endings), as well as blood vessels, nerve fibers, sebaceous and sweat glands and hair follicles.

The epidermis is made up of the viable epidermis and the outermost layer. The epidermis contains 4 distinct histological layers, composed of keratinocytes, which are stacked on top of each other. The keratinocytes develop at the bottom of the epidermis and rise to the surface, where they are shed as dead, hard, flattened cells. Each sub layer in the epidermis is characterized by a different stage of mitosis (dividing cells at the inner layer to dead cells at the outer layer). This layer is thus constantly being renewed. Melanocytes and Langerhans cells are other important cells of the epidermis. The deeper cells of the epidermis (Stratum Granulosum) retain their desmosomal junctions, but as they are pushed to the surface by newly forming cells of the Stratum Spinosum(SS), the dead cells gradually break apart and are lost, a process called desquamation. The dead and dying cells filled with mature keratin form the stratum corneum (SC) are surrounded by a lipid layer, and the SC is regarded as the magor barrier for transdermal permeation. The SC structure has inspired theoretical researchers to apply a model of “bricks and mortar” when trying to simulate and/ or anticipate skin permeability.

As the skin is the actual wrap of the body organs, it has to function as a selective wrap, allowing controlled “matter flow” through it. As the SC is the outer layer of the skin—it is functioning as the most important barrier for this matter flow. The barrier property of the human skin is vital for limiting the body uptake or loss of foreign and endogenous substances. Most compounds (except for the most lipophilic ones) with octanol/water partition coefficient 1-100, with a molecular weight of less than 500 g/mol, require at least 2 hours to penetrate 10-15 um into the skin (despite the fact that it is permeable!), translated into permeability (P) of less than 10⁻⁴ cm/sec¹. This remarkable impregnation capability is related to the heterogeneous internal structure of the SC. As previously mentioned, the SC consisted of keratinized coenocytes which are closely packed in ten layers—thus performing as a geometrical barrier. The close packing reduces the diffusional spaces—thus increasing the diffusion path length. Once this geometrical barrier is passed—a permeant permeation is blocked within the extra cellular matrix spaces by a continuous phase of an extended lipid bilayer, composed primarily of fatty acids, cholesterol and spingolipds. The permeability of a permeant into the body and through the skin is governed by the relative permeability through both layer of the SC: The shape and the arrangement of the geometrical barrier and the extent of permeant partitioning at the lipid bilayers.

Besides the physical barrier possessed by the skin structure, the body also retains immunological and histological responses (Langerhans cells) as well as enzymes which will be activated once a substance is permeating through the physical barrier. Therefore—even those molecules that manage to diffuse through the skin layers, are expected to be quickly metabolized.

This protective functionality of the skin presents a challenge—once we do want the transdermally introduce a compound into the body (e.g. active reagents of various types—including drugs, cosmetic ingredients and so on).

The process of introducing a permeant into the blood stream (which is the desired goal in most drugs), can be separated into many stages—each of them possessing a barrier: the diffusion from one layer to the other will dramatically be depended on the partition of the permeant between the outer layer and the targeted inner layer. Taking into account that the skin can be divided into at least 6 distinct layers—each passage from one layer to another is a challenge. The success in this process depends also on the permeant chemical nature, the formulation it is applied in and the physical properties of such a formulation (such as viscosity, enhancers, micelles presence). The actual permeation into the tissue can occur through 3 routes: through the appendages (hair follicles, sweat ducts) also referred to as shunt routes (only 0.1% of skin surface), through the intercellular lipid domains (diffusion), or through transcellular route. There are various approaches applied in order to overcome the barrier in trans dermal permeation—each one suffering from other faults.

The classical approach towards TTS (trans therapeutic system) design was to use permeant releasing patches, thus controlling the exposed area, amount applied and release kinetics and release duration. How ever, this approach does not improve permeability, but rather—maximizes the permeation based on given parameters.

There are also approaches than are designed to increase permeability into the tissue, using various means:

One of the approaches to resolve transdermal permeation is using Iontophoretic processes where electrical charge is applied in order to drive molecules through the SC. Another approach relays on using a multi needles devices, aiming to reduce the pain for patient compliance—while creating multi tunneling effect for direct administration of the permeant.

A complete different approache that is used to enlarge the number of transdermally-applicable drugs utilizes permeation enhancers/promotors. These compounds promote drug permeation through the skin by a reversible decrease of the barrier resistance. Enhancers can act on the stratum corneum intracellular keratin, influence desmosomes, modify the intercellular lipid domains or alter the solvent nature of the stratum corneum. Even though hundreds of substances have been identified as permeation enhancers to date, yet our understanding of the structure-activity relationships is limited. In general, enhancers can be divided into two large groups: small polar solvents, e.g. ethanol, propylene glycol, dimethylsulfoxide and amphiphilic compounds containing a polar head and a hydrophobic chain (surfactants), e.g. fatty acids and alcohols, 1-dodecylazepan-2-one (Azone), 2-nonyl-1,3- dioxolane (SEPA 009), and dodecyl-2-dimethylaminopropanoate (DDAIP). Amphiphilic compounds, or as they are more commonly known—surfactants/soaps, have two distinct properties that allow them to perform as enhancers: (1) They are composed of a hydrophobic tail (of chosen length and effect) and a hydrophilic head [varying between positively charged salts (cationic surfactants), negatively charged salts (anionic surfactants) and polar functional groups (non-ionic surfactants)]. (2) Above the critical micelle concentration (CMC), they form a defined micellar structure—which serves as a potential carrier pocket for other molecules.

There are various advantages of using peptides as the carrier of a drug molecule. Among those one should consider the low proteolytic activity of the skin which increases the stability of the carrier during permeation; the safety and biocompatibility expected from a sequences derived from a human protein; The fact that the carrier is biodegradable with high probability of biocompatible degradation products.

Previous academic work on bacterial membrane penetration of peptides was focused on membrane disrupting peptides. These peptides are capable of forming pores in the membrane by self assembling to the membrane. Thus, these peptides are a potential source of antibiotics since they are capable of perturbing the bacterial membrane to a state of no osmotic pressure. However, these peptides are not capable of penetrating skin since the stratum corneum consists of about 100 multilamellar lipid bilayers, unlike the single bilayer found in a bacterial cell. However, magainin, a 23 aa peptide was found to disrupt liposome vesicles made from lipids representative of those found in human stratum corneum¹. In a later paper it was shown that the magainin synergistically enhances the transdermal penetration of a labeled drug molecule in the presence of N-Lauroyl Sarcosine (NLS)².

There are few papers published in the last decade on the use of peptides in transdermal drug delivery. In 2006 Chen et al. published a paper in Nature Biotechnology, describing a peptide chaperone which enhanced both dimer and hexamer insulin transdermal penetration³. Other works have shown that PTDs (protein transduction domain) peptides can carry covalently conjugated compounds and penetrate the skin of living animals^{4,5,6,7,8}. In 2007 a work published by Chen, Lee et al.⁹ presented a significant enhancement of GFP (green fluorescent protein) permeation through mouse skin in the presence of various peptidic sequences. The peptides were not covalently bound to the protein and thus this work is important in the sense that, although the mechanism was not resolved—the potential coordination of peptide-molecule is demonstrated to be effective in the permeation process. This observation is supported by another publication from 2008, in which the Botulinium Toxin type A was transdermally transported in the presence of a MTS (Macromolecule Transport System) based on peptides¹⁰. In this publication and based on previous publications—the dermal permeation enhancement of the MTS is related to two dominant routes: 1) Energy dependant transcytosis shuttle of peptides trough the cells, and 2) Energy independent diffusion of the peptide carriers through the fluid sections of the membrane. The authors claim that the ampiphilic structure of the peptides plays a crucial role in their ability to enhance the transdermal penetration of the toxin.

Several papers were published on a group of peptides, derived from a fibrinogen, which are capable of cell binding¹¹, through what appears to be a non selective, non receptor related mechanism of binding. In a later paper, these haptides were shown to induce a transcutaneous delivery of HR-gp 100 protein¹². These researchers speculated that the relatively high content of charged amino acids in the haptides induce a non-specific polarization of the cell membrane, thus creating tunnels into the cell. This property can explain both the cell binding [11] as well as the transdermal permeation of the HR-gp100 protein [12].

In another work it was demonstrated that arginine rich peptides are capable of transdermally transporting a protein into living cells without being covalently bound to the protein¹³.

The ficolins form a group of proteins having collagen-, and fibrinogen-like domains. They were first identified as proteins that bind to TGF-β1. Three types of ficolin have been identified in humans: L-ficolin, H ficolin and M ficolin. A ficolin polypeptide consists of a small N-terminal domain, a collagen-like domain, a neck region, and a fibrinogen-like domain, which shows similarity to the C-terminal halves of the beta and gamma chains of fibrinogen. The collagen-like domain mediates the association of ficolin polypeptides into trimers, and the N-terminal domain contains cysteine residues which permit the covalent assembly of trimers into higher oligomers with a “bouquet-like” appearance. This supramolecular organization resembles that of the collectins, a group of C-type lectins which have a C-type CRD in place of the fibrinogen-like domain found in ficolins. Collectins and ficolins are also functionally similar. The collectin mannose binding protein (MBP) is a serum host defense protein in which the C-type CRDs recognize arrays of GlcNAc and mannose residues on pathogen surfaces. MBP initiates the lectin branch of the complement system via activation of MBP-associated proteases (MASPs), leading to elimination of the target pathogen. Two of the three human ficolins, ficolins L and H, are also serum proteins which bind to pathogen surfaces via interaction with carbohydrates (and probably with other molecules), and trigger complement activation though association with MASPs. Ficolin L also acts as an opsonin, promoting phagocytosis of pathogens by neutrophils. Ficolin L polymorphisms affect serum protein levels and sugar binding and may have pathophysiological implications. The third human ficolin, ficolin M, is found in secretory granules in neutrophils and monocytes, recognizes pathogens in a carbohydrate-dependent manner and activates complement via MASPs. Ficolin M may also act as a phagocytic receptor. Ficolins L and H are produced in the liver, in common with MBP, and ficolins M and H are produced in the lung, like the antimicrobial collectins SP-A and SP-D. Human ficolins and MBP also participate in the recognition and clearance of apoptotic cells. Two ficolins, A and B, are present in mouse. Ficolin B is found in the lysosomes of activated macrophages and is suggested to be the ortholog of ficolin M, but it appears that only ficolin A is associated with MASPs and can activate complement. The mouse ortholog of ficolin H is a pseudogene.

International Patent Application PCT/IL2009/001025 discloses a tissue adhesive comprising ficolin subsequences. International Patent Application 2008/001121 discloses pharmaceutical compositions comprising ficolin subsequences for sequestering cells in connective tissue.

### SUMMARY OF THE INVENTION

In its first aspect, the present invention provides novel surfactants. The surfactants of the invention are peptide sequences derived from ficolin. The peptide sequences of the invention may have a length, for example, in the range of 5 to 20 amino acids, or in the range of 10 to 20 amino acids, or in the range of 10 to 15 amino acids. The ficolin may be a human ficolin, and as such is non-toxic and biocompatible. In one embodiment, the peptide sequences are derived from the so-called “tethered arms” of the ficolin molecule.

Examples of surfactants of the invention include the following peptides, all of which are subsequences of the tethered arms of human ficolin:
(a) the peptide, referred to herein as “C-Fic” having the sequence KGYNYSYKSEMKVRPA, and having the SEQ. ID. No. 1;
(b) the peptide, referred to herein as “M-Fic” having the sequence GGWTVFQRRVDGSVDFYRK, and having the SEQ. ID. No. 2.
(c) the peptide, referred to herein as “C-M-Fic” having the sequence KGYNYSYKVSEMKFQRRVDGSVDFYRK, and having the SEQ. ID. No. 3.
(d) the peptide, referred to herein as “C-Fic-a-K” having the sequence KGYKYSYKVSEMKVRPAK, and having the SEQ. ID. No. 4;
(e) the peptide, referred to herein as “M-Fic-K” having the sequence GGWTVFQRRMDGSVDFYRK, and having the SEQ. ID. No. 5;
(f) the peptide, referred to herein as “C-M-Fic2K” having the sequence KGYKYSYKGGWTVFQRRMDGSVDFYRK, and having the SEQ. ID. No. 6;
(g) the peptide, referred to herein as “C-M-Fic-a-K” having the sequence KGYKYSYKVSEMKFQRRMDGSVDFYRK, and having the SEQ. ID. No. 7; and
(h) the peptide, referred to herein as “C-M-Fic2” having the sequence KGYKYSYKGGWTVFQRRMDGSVDFYR, and having the SEQ. ID. No. 8.

In another of its aspects, its aspects, the invention provides a pharmaceutical composition comprising one or more surfactants of the invention. The inventors have further found that the pharmaceutical composition of the invention promotes permeation of small molecules and large molecules through body surfaces, such as skin. Thus, the pharmaceutical composition of the invention may comprise, in addition the surfactant, one or more molecules to be delivered through a body surface. In particular, the pharmaceutical composition may be used for percutaneous delivery into the body. Without wishing to be bound by a particular theory, it is believed that in the case of the enhancement of transdermal permeability of small molecules, the surfactants of the invention form micelles in high concentrations, thus becoming efficient carrier for small molecules. It is believed that enhancement of the permeation of large molecules in this case the surfactants tend to coordinate with the large molecules, while inducing a reversible change on the stratum corneum.

The pharmaceutical compostion can be in any form. In one embodiment, the pharmaceutical composition is in a form suitable for application to a body surface, such as a cream or lotion.

In another of its aspects, the invention provides a peptide sequence derived from ficolin for use as a surfactant.

The invention also provides a peptide sequence derived from ficolin for use in the preparation of a pharmaceutical composition for delivery of a substance into a body surface.

In yet another of its aspects, the invention provides a method for percutaneous delivery of one or more substances into a body surface. The method of the invention comprises applying to the body surface a pharmaceutical composition of the invention comprising the one or more substances.

Thus, in one of its aspects, the invention provides use of a peptide having a sequence that is a subsequence of a ficolin protein as a surfactant. The peptide may comprise, for example, at least 5 amino acids, or at least 10 amino acids. The peptide may be a subsequence of human ficolin. The peptide derived from a tethered arm of the ficolin molecule.

The peptide may be selected from the group comprising:
(a) the peptide, referred to herein as “C-Fic” having the sequence KGYNYSYKSEMKVRPA, and having the SEQ. ID. No. 1;
(b) the peptide, referred to herein as “M-Fic” having the sequence GGWTVFQRRVDGSVDFYRK, and having the SEQ. ID. No. 2.
(c) the peptide, referred to herein as “C-M-Fic” having the sequence KGYNYSYKVSEMKFQRRVDGSVDFYRK, and having the SEQ. ID. No. 3.
(d) the peptide, referred to herein as “C-Fic-a-K” having the sequence KGYKYSYKVSEMKVRPAK, and having the SEQ. ID. No. 4;
(e) the peptide, referred to herein as “M-Fic-K” having the sequence GGWTVFQRRMDGSVDFYRK, and having the SEQ. ID. No. 5;
(f) the peptide, referred to herein as “C-M-Fic2K” having the sequence KGYKYSYKGGWTVFQRRMDGSVDFYRK, and having the SEQ. ID. No. 6;
(g) the peptide, referred to herein as “C-M-Fic-a-K” having the sequence KGYKYSYKVSEMKFQRRMDGSVDFYRK, and having the SEQ. ID. No. 7; and
(h) the peptide, referred to herein as “C-M-Fic2” having the sequence KGYKYSYKGGWTVFQRRMDGSVDFYR, and having the SEQ. ID. No. 8.

In another of its aspects, the invention provides a pharmaceutical composition comprising a surfactant wherein the peptide has a sequence that is a subsequence of a ficolin protein as a surfactant. The peptide may comprise at least 5 amino acids, at least 10 amino acids, or at least 20 amino acids. The peptide may be a subsequence of human ficolin. The peptide may be derived from a tethered arm of the ficolin molecule.

The peptide may be selected from the group comprising:
(a) the peptide, referred to herein as “C-Fic” having the sequence KGYNYSYKSEMKVRPA, and having the SEQ. ID. No. 1;
(b) the peptide, referred to herein as “M-Fic” having the sequence GGWTVFQRRVDGSVDFYRK, and having the SEQ. ID. No. 2.
(c) the peptide, referred to herein as “C-M-Fic” having the sequence KGYNYSYKVSEMKFQRRVDGSVDFYRK, and having the SEQ. ID. No. 3.
(d) the peptide, referred to herein as “C-Fic-a-K” having the sequence KGYKYSYKVSEMKVRPAK, and having the SEQ. ID. No. 4;
(e) the peptide, referred to herein as “M-Fic-K” having the sequence GGWTVFQRRMDGSVDFYRK, and having the SEQ. ID. No. 5;
(f) the peptide, referred to herein as “C-M-Fic2K” having the sequence KGYKYSYKGGWTVFQRRMDGSVDFYRK, and having the SEQ. ID. No. 6;
(g) the peptide, referred to herein as “C-M-Fic-a-K” having the sequence KGYKYSYKVSEMKFQRRMDGSVDFYRK, and having the SEQ. ID. No. 7; and
(h) the peptide, referred to herein as “C-M-Fic2” having the sequence KGYKYSYKGGWTVFQRRMDGSVDFYR, and having the SEQ. ID. No. 8.

The pharmaceutical composition of the invention may further comprising an active ingredient for delivery through a body surface. The pharmaceutical composition may be in a form suitable for topical application to a body surface, such as a lotion or cream. The body surface may be, for example, a skin surface.

The invention also provides a method for penetration of a substance through a body surface comprising applying to the body surface a pharmaceutical composition of the invention. The body surface may be, for example, a skin surface.

### BRIEF DESCRIPTION OF THE DRAWINGS

In order to understand the invention and to see how it may be carried out in practice, embodiments will now be described, by way of non-limiting examples only, with reference to the accompanying drawings, in which:

FIG. 1 shows transport of hyaluronic acid through pig skin;

FIG. 2 shows the toxicity of the peptides C-Fic, M-Fic, and M-C-Fic to FF1 cells;

FIG. 3 shows toxicity of the peptides C-Fic, M-Fic, and M-C-Fic to BAEC cells;

FIG. 4 shows the toxicity of various peptides to FF1 cells after 5 days of incubation; and

FIG. 5 shows the toxicity of various peptides to BAEC cells after 5 days of incubation.

### EXAMPLES

Skin samples (stored at −80° C.) were thawed at 4° C. for approximately 15 hr followed by 1 hr at room temperature prior to use. A thawed pig dermatomed skin sample was mounted horizontally on a Franz cell, dermis side down. The Franz cell was connected to a 37° C. circulating water bath, yielding a tissue temperature of 32° C., comparable to the physiological temperature of the skin surface. The receptor phase consisted of PBS pH 7.4 with continuous stirring.

A test formulation, formulated as a suspension was applied to the skin and uniformly dispersed over the skin surface at a surface density of at least 1.0 g/cm² to insure pseudo-infinite dose conditions. The formulations consisted of a peptide surfactant of the invention together with hyaluronic acid. The formulations used were as described in the following table:

| Formulation | |
|---|---|
| number | Formulation description |
| 1 | 5 mg/ml HA, (control) |
| 2 | 5 mg/ml HA + 60 mg/ml CMfick |
| 3 | 5 mg/ml HA + 2% N Lauroyl Sarcosine Sodium salt (NLS) |
| 4 | 5 mg/ml HA + 60 mg/ml Mfick |

The dose chamber was sealed with an occlusive film to prevent loss of any volatile component of the test formulation. 200 μl aliquots of the receptor phase were collected at t=1 hr, 3 hrs, 5 hrs and 7 hrs following the application of the formulation to the skin. Each aliquot was replaced with an equal volume of PBS.

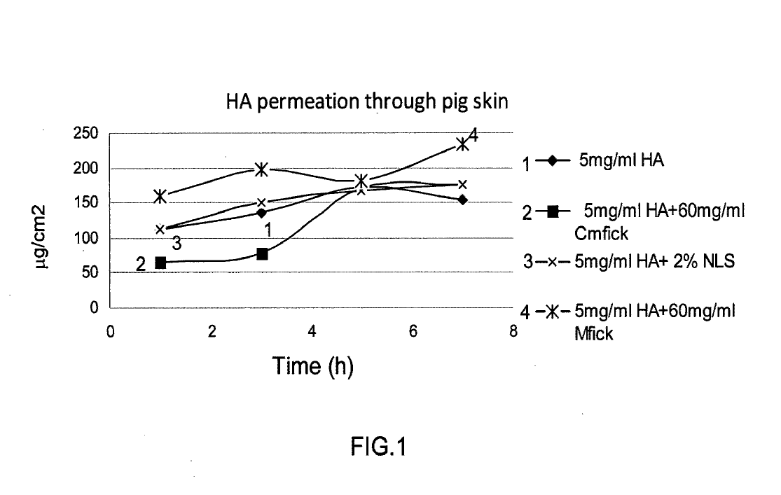

The samples were analyzed for the presence of hyaluronic acid by the Carbazole method as is known in the art [13]. The results are shown in FIG. 1. in control experiments in which the surfactant was omitted (curve 1 in FIG. 1), about 40 ?g/cm² HA traversed the skin in a period of 6 hours. In the presence of 2% NLS (curve 3 in FIG. 1), about 65 ?g/cm² traversed the skin during the same time period. In the presence of CMfick (curve 2) and Mfick (curve 4), 110 ?g/cm² and 80 ?g/cm², respectively, traversed the skin during this time period.

Toxicity assay for the different peptides:

A toxicity assay was done to determine the toxicity of the peptides tested to either one of the cell lines used. 15×10³ cells bovine aortal endothelial cells (BAEC) and human foreskin fibrobalsts (FF1) were seeded in 96 well plastic plates. After an overnight incubation, increasing concentrations of peptides in the range of 0.1-300 μg/ml were added to the wells. Cell survival was checked by the MTS assay after 2 and 5 days and normalized to the cell number of the controls (no peptide).

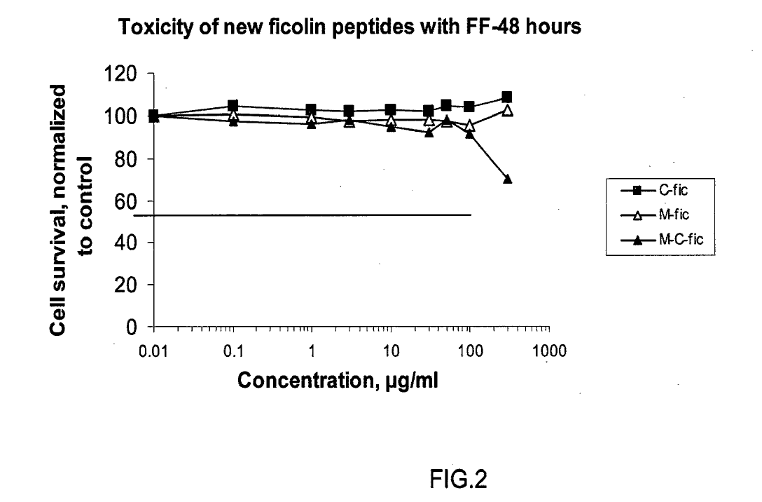

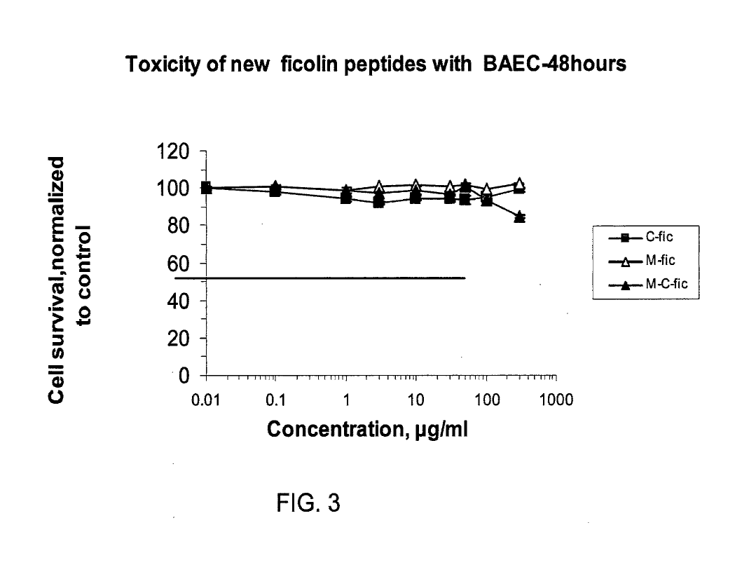

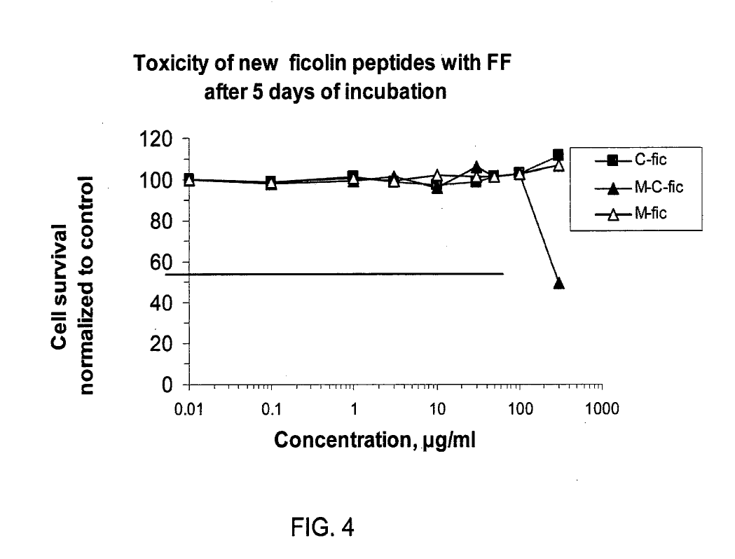

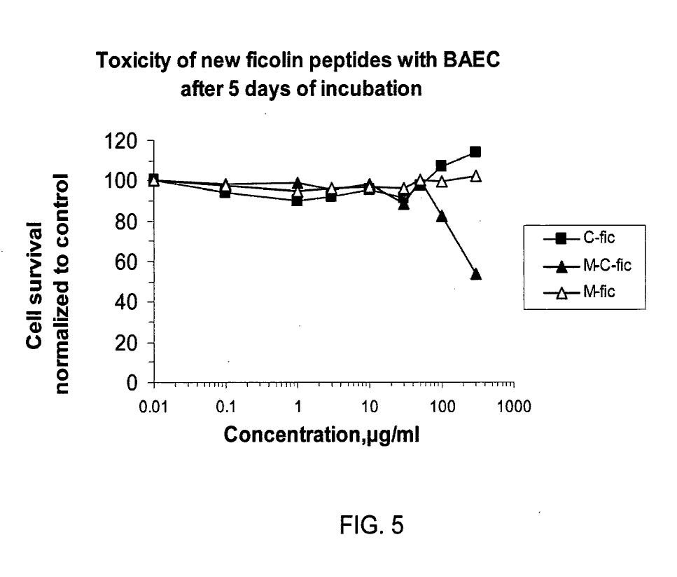

The toxicity of C-Fic, M-Fic, and M-C-Fic to FF1 cells is shown in FIG. 2 and the toxicity of C-Fic, M-Fic, and M-C-Fic to BAEC cells is shown in FIG. 3 (FIG. 13b). The graphs percent of cells relative to the control in which the peptide was omitted, over a wide range of peptide concentration, after 48 hours. The toxicity of the various peptides to FF1 and BAEC cells after 5 days of incubation is shown in FIGS. 4 and 5 respectively.

1-19. (canceled)

20. A method for enhancing transdermal permeation of a substance, comprising applying to a body surface, a therapeutically effective amount of a peptide having an amino acid sequence that is a subsequence of a ficolin protein.

21. The method according to claim 20, wherein the peptide comprises at least 5 amino acids.

22. The method according to claim 21, wherein the peptide comprises at least 10 amino acids.

23. The method according to claim 20, wherein the ficolin protein is human ficolin.

24. The method according to claim 20, wherein the peptide is derived from a tethered arm of the ficolin molecule.

25. The method according to claim 20, wherein the peptide is selected from the group consisting of

| | |
|---|---|
| KGYNYSYKSEMKVRPA, | (SEQ ID NO: 1) |
| GGWTVFQRRVDGSVDFYRK, | (SEQ ID NO: 2) |
| KGYNYSYKVSEMKFQRRVDGSVDFYRK, | (SEQ ID NO: 3) |
| KGYKYSYKVSEMKVRPAK, | (SEQ ID NO: 4) |
| GGWTVFQRRMDGSVDFYRK, | (SEQ ID NO: 5) |
| KGYKYSYKGGWTVFQRRMDGSVDFYRK, | (SEQ ID NO: 6) |
| KGYKYSYKVSEMKFQRRMDGSVDFYRK, | (SEQ ID NO: 7) |
| and | |
| KGYKYSYKGGWTVFQRRMDGSVDFYR. | (SEQ ID NO: 8) |

26. A pharmaceutical composition for enhancing transdermal permeation comprising a peptide having a sequence that is a subsequence of a ficolin protein.

27. The pharmaceutical composition according to claim 26, wherein the peptide comprises at least 5 amino acids.

28. The pharmaceutical composition according to claim 26, wherein the peptide comprises at least 10 amino acids.

29. The pharmaceutical composition according to claim 26, wherein the peptide comprises at least 20 amino acids.

30. The pharmaceutical composition according to claim 26, wherein the ficolin protein is human ficolin.

31. The pharmaceutical composition according to claim 26, wherein the peptide is derived from a tethered arm of the ficolin molecule.

32. The pharmaceutical composition according to claim 26, wherein the peptide is selected from the group consisting of

| | |
|---|---|
| KGYNYSYKSEMKVRPA, | (SEQ ID NO: 1) |
| GGWTVFQRRVDGSVDFYRK, | (SEQ ID NO: 2) |
| KGYNYSYKVSEMKFQRRVDGSVDFYRK, | (SEQ ID NO: 3) |
| KGYKYSYKVSEMKVRPAK, | (SEQ ID NO: 4) |
| GGWTVFQRRMDGSVDFYRK, | (SEQ ID NO: 5) |
| KGYKYSYKGGWTVFQRRMDGSVDFYRK, | (SEQ ID NO: 6) |
| KGYKYSYKVSEMKFQRRMDGSVDFYRK, | (SEQ ID NO: 7) |
| and | |
| KGYKYSYKGGWTVFQRRMDGSVDFYR. | (SEQ ID NO: 8) |

33. The pharmaceutical composition according to claim 26, further comprising an active ingredient for delivery through a body surface.

34. The pharmaceutical composition according to claim 33, in a form suitable for topical application to a body surface.

35. The pharmaceutical composition according to claim 34, in the form of a lotion or cream.

36. The pharmaceutical composition according to claim 34, wherein the body surface is a skin surface.

37. A method for penetration of a substance through a body surface, comprising applying to the body surface a therapeutically effective amount of a pharmaceutical composition according to claim 26.

38. The method according to claim 37, wherein the body surface is a skin surface.
